(19) United States
(12) Patent Application Publication
(10) Pub. No.: US 2004/0111005 A1
(43) Pub. Date: Jun. 10, 2004

(54) Magnetic accessory

(76) Inventor: Johnny M. J. Lu, New York, NY (US)

Correspondence Address: SUPREME PATENT SERVICES, POST OFFICE BOX 2339, SARATOGA, CA, 95070, US

(21) Appl. No.: 10/313,134

(22) Filed: Dec. 6, 2002

(51) Int. Cl. A61N 1/00

(52) U.S. Cl. 600/15

(57) ABSTRACT

A magnetic accessory includes a number of magnetic stones and sets of beads alternating each other in a line. The magnetic stones and the sets of beads are secured to each other to form a chain. The magnetic accessory can be worn by surrounding the wearer's body, such as neck, waist, ankle and wrist and secured to the wearer's body by the magnetic stones magnetically attracting each other. Thus, the magnetic accessory can be worn in a variety of ways by attaching different magnetic stones together. The magnetic stones that are worn in the wearer' body induce a magnetic field inside the wearer's body for healing purposes to improve blood circulation, strengthen revitalization of the nerves, eliminate aches and soreness in shoulder and waist areas and relieve stiffness and fatigue. In addition, the magnetic stone can serve as fasteners that retain a decorative member having a ferrous portion thereon and allows for readily change of the decorative member.

### FIELD OF THE INVENTION

The present invention relates generally to a magnetic accessory, and in particular to a chain comprised of magnetic stones alternating with decorative beads.

### BACKGROUND OF THE INVENTION

Magnetic field induced on predetermined points of human body for healing purposes has been long known. The healing power of the magnetic field induced inside human body has been proven for thousands of years. Acupuncture is an example of the therapy of magnetic field. Another way to apply a magnetic field to a human body is to tap magnets on the predetermined points of the human body. The magnets induce a magnetic field penetrating into the human body and revitalizing ions in human body to activate blood circulation. Thus, a number of devices that comprise magnets to induce magnetic field in human body are available in the market. However, most of the devices are single function devices. Namely, they provide healing function only.

Accessories or jewelries are often used by ladies for beautifulness. Necklaces and bracelets are some of the most commonly adapted accessories. The necklaces and bracelets comprise a chain comprised of a number of beads, such as pearls, lined up and secured by a thread or wire extending through all the beads. A releasable fastener is provided on ends of the chain to secure the ends together. Certain steps must be taken in securing and/or releasing the ends of the chain. This may sometimes be troublesome to the wearer for the fastener is very tiny. In addition, the chain is only attached to the human body, such as neck or wrist, by securing the ends thereof together. This makes it inflexible in wearing the chain. Variation in the way of wearing the chain is almost impossible.

Thus, it is desired to provide a magnetic accessory for overcoming the above deficiencies.

### SUMMARY OF THE INVENTION

An object of the present invention is to provide a magnetic accessory comprising a number of beads and magnetic stones fixed together as a chain wherein the magnetic stones are arranged in an equally spaced manner for providing healing power to a wearer.

Another object of the present invention is to provide a magnetic accessory comprising a number of magnetic stones equally spaced among beads that are lined up and secured as a chain, the magnetic stones being magnetically attractive to and thus releasably attached to each other for mounting the magnetic accessory to a wearer and the way of wearing being variable by differently attaching the magnetic stones to each other.

A further object of the present invention is to provide a magnetic fastener for jewelries and other similar personal articles that allows for change of a decorative portion of the personal articles.

To achieve the above objects, in accordance with the present invention, there is provided a magnetic accessory comprising a number of magnetic stones and sets of beads alternating each other in a line. The magnetic stones and the sets of beads are secured to each other to form a chain. The magnetic accessory can be worn by surrounding the wearer's body, such as neck, waist, ankle and wrist and secured to the wearer's body by the magnetic stones magnetically attracting each other. Thus, the magnetic accessory can be worn in a variety of ways by attaching different magnetic stones together. The magnetic stones that are worn in the wearer' body induce a magnetic field inside the wearer's body for healing purposes to improve blood circulation, strengthen revitalization of the nerves, eliminate aches and soreness in shoulder and waist areas and relieve stiffness and fatigue. In addition, the magnetic stone can serve as fasteners that retain a decorative member having a ferrous portion thereon and allows for readily change of the decorative member.

### BRIEF DESCRIPTION OF THE DRAWINGS

The present invention will be apparent to those skilled in the art by reading the following description of preferred embodiments thereof, with reference to the attached drawings, in which:

FIG. 1 is a perspective view of a magnetic accessory constructed in accordance with the present invention in the form of a chain;

FIG. 2 is a plan view of a magnetic accessory in accordance with another embodiment of the present invention formed by positioning a number of chains of FIG. 1 side by side;

FIG. 3 is a schematic view showing a first example of wearing the magnetic accessory in a wearer's ankle;

FIG. 4 is a schematic view showing a second example of wearing the magnetic accessory in a wearer's neck;

FIG. 5 is a schematic view showing a third example of wearing the magnetic accessory in a wearer's neck;

FIG. 6 is a schematic view showing a fourth example of wearing the magnetic accessory in a wearer's waist;

FIG. 7 is a perspective view of a magnetic accessory in accordance with a further embodiment of the present invention;

FIG. 8 is an exploded view of a magnetic accessory in accordance with yet a further embodiment of the present invention which is made in the form of a finger ring; and

FIG. 9 is an exploded view of a magnetic accessory in accordance with a further embodiment of the present invention that is made in the form of a neck tie tack.

### DETAILED DESCRIPTION OF THE INVENTION

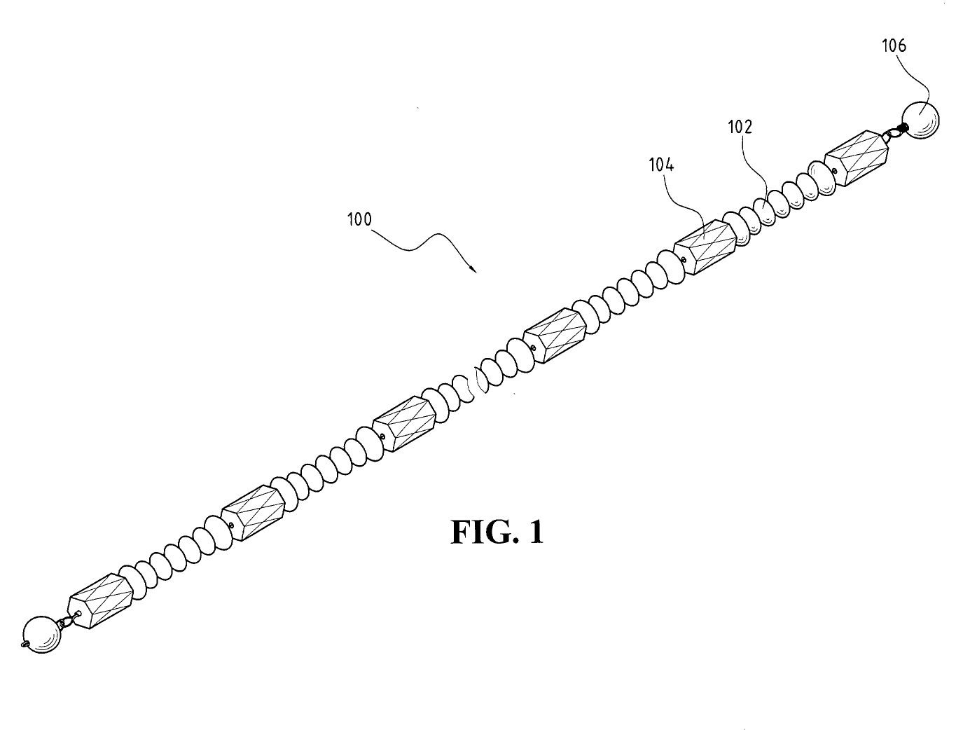

With reference to the drawings and in particular to FIG. 1, a magnetic accessory constructed in accordance with the present invention, generally designated with reference numeral 100, comprises a number of sets of beads 102, such as pearls and colorful balls, arranged in a line and a number of magnetic stones 104 arranged in the line of the beads 102 in such a way that the magnetic stones 104 and the bead sets alternate each other and the magnetic stones 104 are equally spaced from each other. In other words, the number of beads 102 located between any adjacent magnetic stones 104 is the same. The beads 102 and the magnetic stones 104 are secured together to form a chain having opposite ends. If desired, end beads 106 may be attached to each end of the chain. The end beads 106 may be the same as the beads 102 or they can be different.

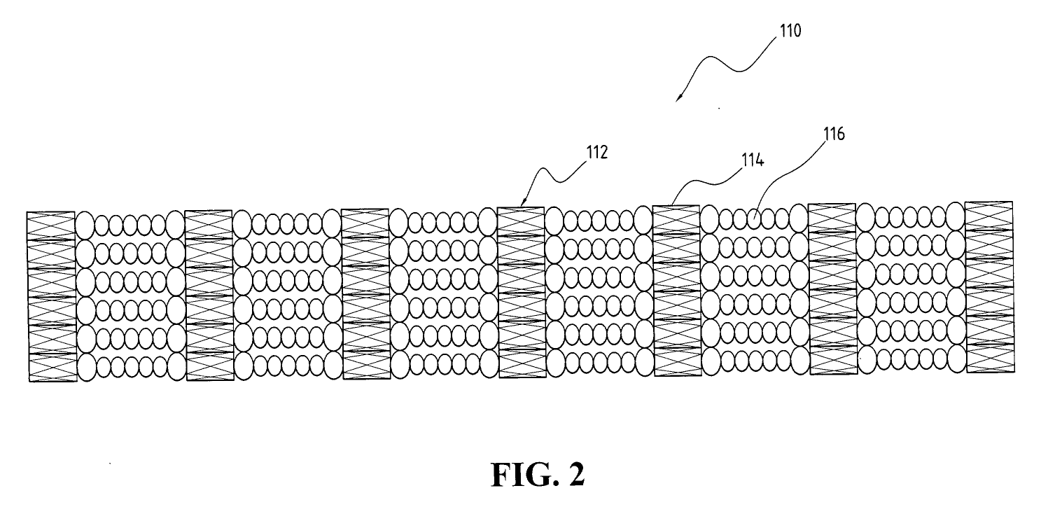

FIG. 2 shows a different embodiment of the magnetic accessory in accordance with the present invention, which is designated with reference numeral 110 for distinction. The magnetic accessory 110 comprises a number of chains 112 that are in general similar to the chain of the embodiment with reference to FIG. 1, and are arranged side by side. Each chain 112 comprises a number of magnetic stones 114 spaced from each other by a predetermined number of beads 116 and the magnetic stones 114 and the beads 116 are secured together in a line to form the chain 112.

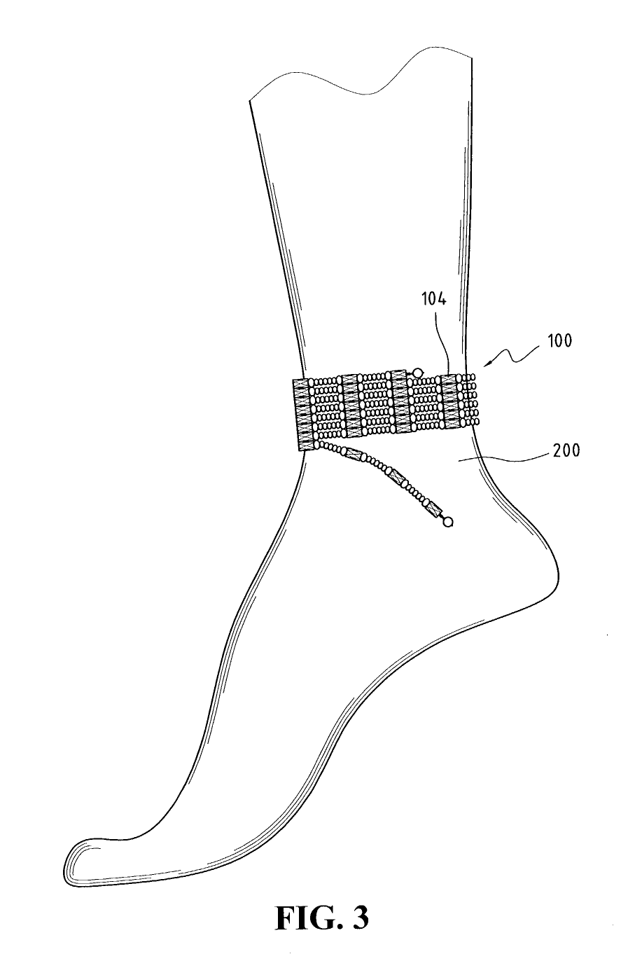

The magnetic force between two magnetic stones 104, 114 allows the magnetic accessory 100, 110 to be attached to itself in a variety of different ways. Thus, mounting the magnetic accessory 100, 110 to a wearer's body can be accomplished in a variety of ways. FIG. 3 shows a first example of mounting the magnetic accessory 100 to the wearer's ankle 200. The chain of the magnetic accessory 100 surrounds the ankle 200 in several turns with the magnetic stones 104 magnetically attached to each other to secure the magnetic accessory 100 around the ankle 200.

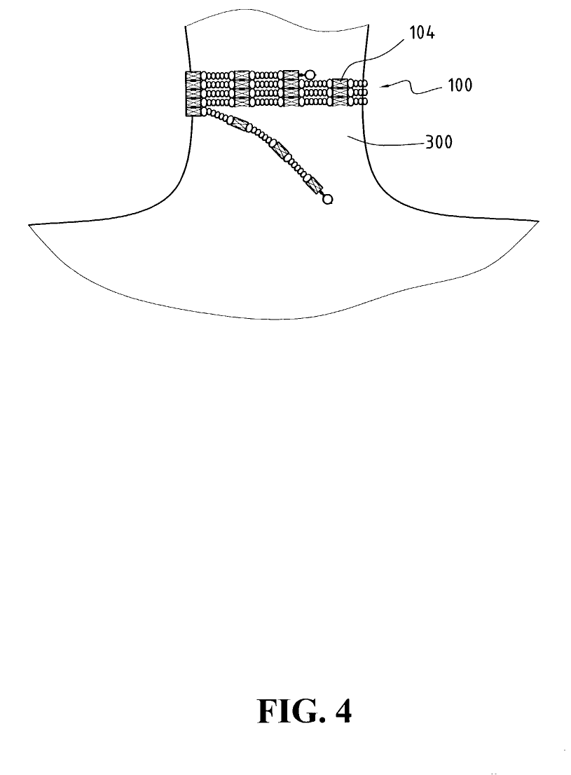

FIG. 4 shows a second example of wearing the magnetic accessory 100 in a wearer's neck 300. Similarly, the chain of the magnetic accessory 100 surrounds the neck 300 several turns with the magnetic stones 104 magnetically attached to each other to secure the magnetic accessory 100 around the neck 300. An end of the chain is allowed to freely move without being secured.

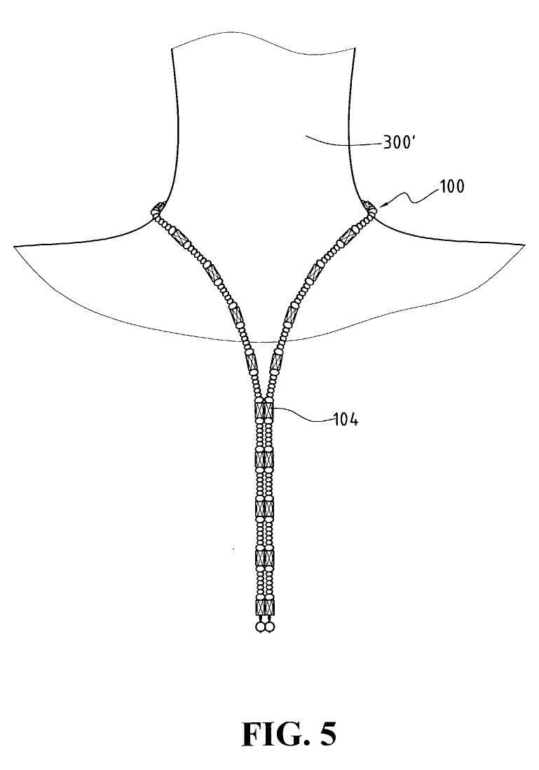

FIG. 5 shows a third example of wearing the magnetic accessory 100 in a wearer's neck 300′. Sections of the chain of the magnetic accessory 100 proximate to the ends thereof are attached to each other by the magnetic attraction between the magnetic stones 104 within these sections. The remaining portion of the chain between the end sections is hung around the neck 300′. This, as compared to the example of FIG. 4, demonstrates the versatility of the chain of the magnetic accessory 100.

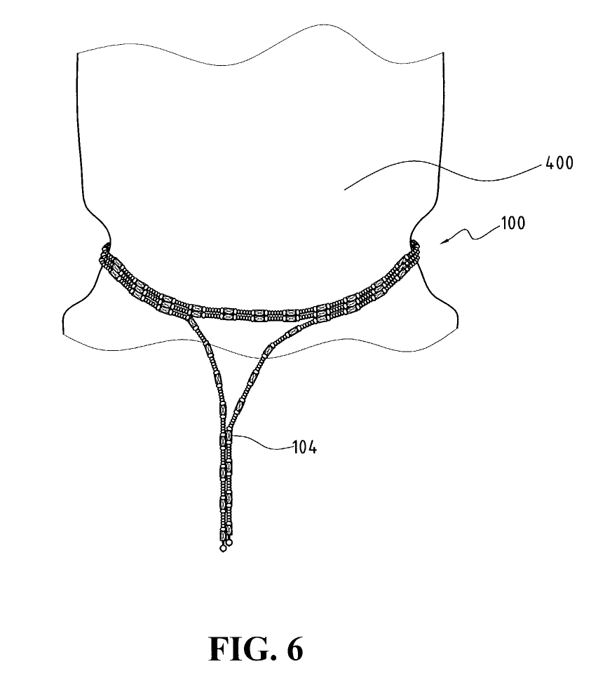

FIG. 6 shows a fourth example of wearing the magnetic accessory 100 around a wearer's waist 400 as a waist belt. The chain of the magnetic accessory 100 surrounds the waist 400 in two turns and the end sections thereof depend down from the waist 400 and are magnetically attached to each other to secure the chain around the waist 400.

The above examples are illustrative ways for wearing the magnetic accessory 100, 110 on a human body. The magnetic accessory 100, 110 so worn makes contact with the human body and the magnetic stones 104, 114 induce a magnetic field penetrating into the human body. The magnetic field helps increasing the number of ions in the blood to improve the blood circulation and strengthening revitalization of nerves. The magnetic field induced inside the human body is also helpful in eliminating or improving aches and soreness in shoulder and waist areas and relieving stiffness and fatigue.

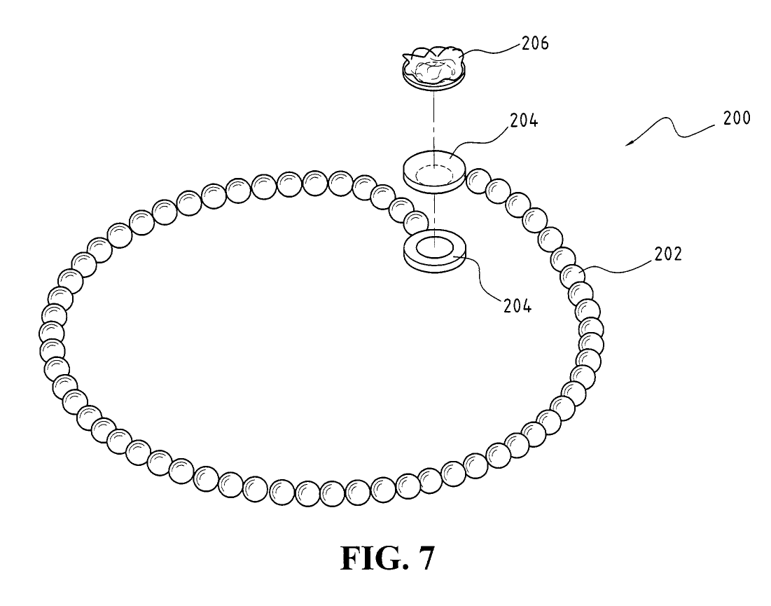

FIG. 7 shows a different embodiment of the magnetic accessory in accordance with the present invention, generally designated with reference numeral 200. The magnetic accessory 200 comprises a chain of beads 202 and two magnetic stones 204 mounted to opposite ends of the bead chain 202. The magnetic stones 204 attract to each and selectively fix the ends of the bead chain 202 together to attach the chain 202 to for example a wearer's neck. A decorative member 206 comprising a ferrous portion or made of a ferrous material is selectively attached to the magnetic stones 204 for decoration purposes. The releasable attachment of the decorative member 206 to the magnetic stones 204 allows the wearer to change different decorative members.

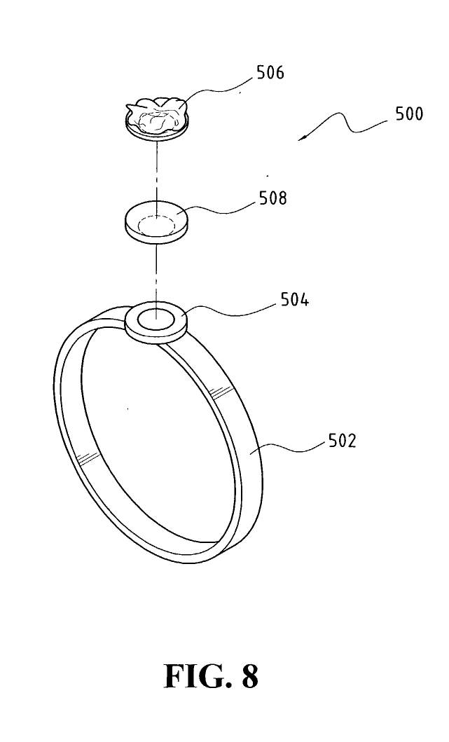

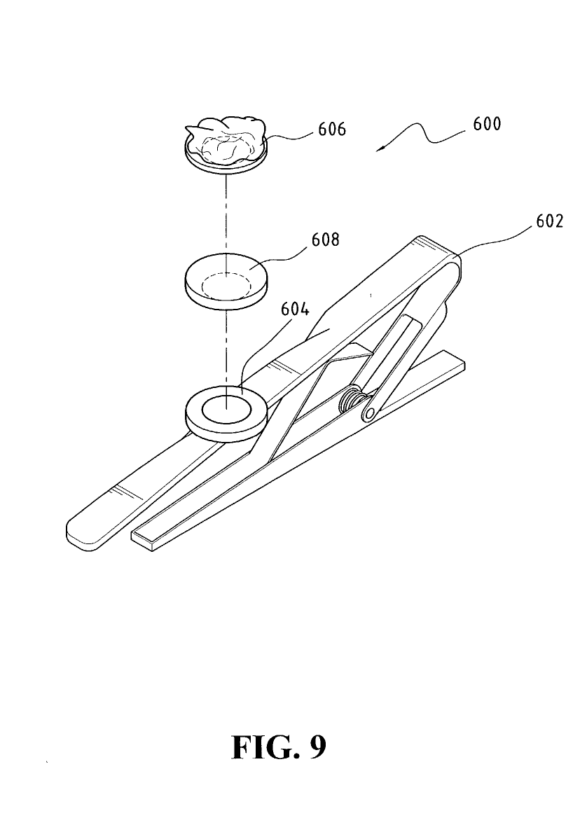

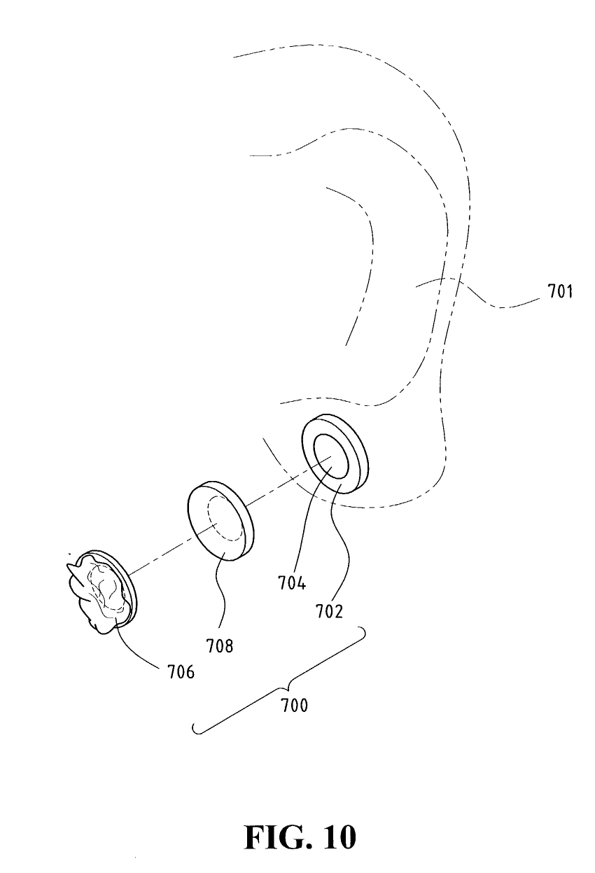

FIG. 8 shows a further embodiment of the magnetic accessory in accordance with the present invention, generally designated with reference numeral 500. The magnetic accessory 500 is embodied as a finger ring, comprising a ring body 502 on which a first magnetic stone 504 is mounted. A decorative member 506 is mounted to a second magnetic stone 508 or alternatively mounted to a ferrous member that is magnetically attracted by and releasably attached to the first magnetic stone 504. The releasable attachment of the decorative member 506 to the first magnetic stone 504 allows the wearer to change different decorative members.

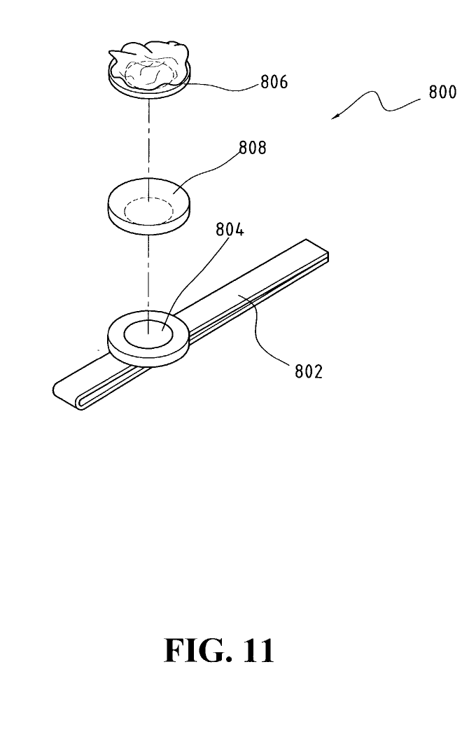

FIG. 9 shows a further embodiment of the magnetic accessory in accordance with the present invention, generally designated with reference numeral 600. The magnetic accessory 600 is embodied as a neck tie tack, comprising a tack body 602 on which a first magnetic stone 604 is mounted. A decorative member 606 is mounted to a second magnetic stone 608 or alternatively mounted to a ferrous member that is magnetically attracted by and releasably attached to the first magnetic stone 604. The releasable attachment of the decorative member 606 to the first magnetic stone 604 allows the wearer to change different decorative members.

Other examples in which the present invention can be embodied include earrings, decorative accessories for shoes, brooches, pendants, waist belts, necklaces, bracelets, ankle chains, cufflink, hair brooches, neckwear accessories, or the like.

Although the present invention has been described with reference to the preferred embodiments thereof, it is apparent to those skilled in the art that a variety of modifications and changes may be made without departing from the scope of the present invention which is intended to be defined by the appended claims. For example, the first magnetic stone of the finger ring 500 or the neck tie tack 600 can be replaced by a ferrous member that attracts the second magnetic stone 508, 608 to attach the decorative member 506, 606 to the ring body 502 or the tack body 602.

What is claimed is:

1. A magnetic accessory comprising a number of magnetic stones and a number of sets of beads alternating each other in a line, the magnetic stones and the sets of beads being secured to each other to form a chain, wherein the magnetic stones are magnetically attached to each other to mount the magnetic accessory to a wearer's body and induce a magnetic field inside the wearer's body for healing purposes.

2. The magnetic accessory as claimed in claim 1, wherein each set of beads comprises the same number of beads lined up in a line.

3. The magnetic accessory as claimed in claim 1, further comprising at least a second chain comprised of magnetic stones and sets of beads alternating and secured to each other, the second chain being attached to the first chain in a side-by-side manner.

4. A magnetic accessory comprising a chain of beads having opposite ends to which magnetic stones are mounted for selectively attaching to each other for mounting the magnetic accessory to a wearer's body.

5. The magnetic accessory as claimed in claim 4, further comprising at least a decorative member comprising a ferrous portion for being selectively and replaceably attached to the magnetic stones.

6. A magnetic accessory comprising a body on which a first magnetic member is mounted and a decorative member mounted to a second member for selectively and magnetically attaching the decorative member to the body.

7. The magnetic accessory as claimed in claim 6, wherein the second member comprises a magnetic stone.

8. The magnetic accessory as claimed in claim 6, wherein the second member comprises a ferrous portion for magnetically attaching to the first magnetic member.
